(19) United States
(12) Patent Application Publication
(10) Pub. No.: US 2025/0025139 A1
(43) Pub. Date: Jan. 23, 2025

(54) CUTTING DEVICE FOR MAKING PLANAR CUTS INTO TISSUE TO PRODUCE TISSUE FORMS HAVING UNIFORM THICKNESS

(71) Applicant: Musculoskeletal Transplant Foundation, Edison, NJ (US)

(72) Inventors: German Eduardo Jurado Rivera, Rahway, NJ (US); Carlos A. Zabala, Edison, NJ (US); Joed Canales, Harrison, NJ (US)

(21) Appl. No.: 18/772,685

(22) Filed: Jul. 15, 2024

Related U.S. Application Data
(60) Provisional application No. 63/528,184, filed on Jul. 21, 2023.

(51) Int. Cl. A61B 10/02; G01N 1/06

(52) U.S. Cl. CPC A61B 10/0266; A61B 2010/0208; G01N 2001/061

(57) ABSTRACT

A cutting device is provided for resizing a tissue sample by enabling a user to make controlled, precise, and consistent cuts into and through the tissue sample. The device includes a body which is assembled from a base plate, a top plate. The base plate has a recess on its surface, and a tissue cavity extending further down within the recess. Tightening thumb screws provided at opposite ends of the body causes the top plate to apply downward pressure on a tissue sample received and held in the tissue cavity. Front and back slots oriented in alignment with one another are provided through front and back walls, respectively, of the base plate. The slots receive a blade therethrough and guide movement of the blade to perform controlled, precise and consistent cutting of the tissue sample placed in the cavity. Methods of using the cutting device are also provided.

### CROSS-REFERENCE TO RELATED APPLICATIONS

The present application claims the benefit of U.S. Provisional Application No. 63/528,184, filed Jul. 21, 2023, the entire disclosure of which is incorporated by reference herein.

### FIELD OF THE INVENTION

The invention described and contemplated herein relates to a device and methods of using same to produce tissue forms having uniform selected thickness. More particularly, the device enables controlled planar cutting into tissue.

### BACKGROUND

Generally, the reshaping of tissue samples, such as those recovered from donors (allogeneic) or patients (autogenic) into desired shapes, sizes, and configurations is accomplished by any of several methods and techniques such as, without limitation, one or more of cutting, shaving, grating, trimming, grinding, molding, curing, dehydration (e.g., air or heat drying, lyophilizing (freeze drying), etc.), contacting with chemical substances, and crosslinking. The methods and techniques selected and applied depend on the desired size and shape of the tissue form and the type of tissue being reshaped. The desired size and shape of the tissue form may sometimes be determined based on the intended use such as the body feature, defect, or injury, to be treated, and the intended outcome.

The type of tissue being reshaped often has properties and characteristics that guide or even determine which processing and reshaping techniques are necessary and applied to produce the desired tissue form. For example, bone tissue may or may not be at least partially demineralized, adipose tissue may or may not be at least partially delipidized, and virtually and tissue type may be at least partially dehydrated. Furthermore, for example, both bone and cartilage tissues, as well as many other tissue types, may be, for example, subject to one or more of cutting, shaving, grating, etc., and, if reshaped into particulate form or other smaller pieces, quantities of such particles or pieces may be molded or otherwise combined or agglomerated to reshape the particles or pieces into a new desired three dimensional shape

Devices and methods of their use which provide improved control and consistency when performing reshaping techniques on tissue are always welcome by practitioners to produce more consistent and precisely shaped tissue forms from tissue samples. The device and methods described and contemplated herein provide such improved control and consistency for performing reshaping of tissue samples.

### BRIEF DESCRIPTION OF THE FIGURES

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIG. 1 is a perspective view of an exemplary cutting device for reshaping tissue samples that shows the body, handle, and blade thereof;

FIG. 2 is an exploded perspective view of the cutting device of FIG. 1;

FIG. 3 is a top plan view of the base plate of the cutting device of FIGS. 1 and 2;

FIG. 4 is a cross sectional view of the base plate of FIG. 3 taken along line A-A and looking in the direction of the arrows;

FIGS. 5A and 5B are top plan views of typical meniscus cartilage tissue samples suitable for reshaping using the cutting device described and contemplated herein;

FIG. 6 is a front elevational view of the base plate of FIG. 3, showing the front slot through the front wall of the base plate for receiving and guiding the blade;

FIG. 7 is an enlarged view of region B of FIG. 6, showing the terminal end of the front slot;

FIG. 8 is an enlarged view of region C of FIG. 6, showing the beginning end portion of the front slot which has a tapered width for more easily inserting the blade therethrough to begin the cutting process;

FIG. 9 is a bottom plan view of the top plate of the cutting device of FIGS. 1 and 2, showing the raised layer protruding from the bottom surface of the top plate; and

FIG. 10 is a front elevational view of the top plate of FIG. 9 which also shows the raised layer protruding from the bottom surface of the top plate.

### DETAILED DESCRIPTION

The cutting device and methods of use thereof described and contemplated herein provide increased control and consistency when cutting a tissue sample to reshape it into a tissue form having desired size and shape. The tissue form may be an intermediate tissue form which is to be subjected to further processing (which may or may not include further reshaping), or a final tissue form having the desired size and shape, as well as other properties and characteristics, and which is ready for packaging, storage, and use. The size and shape of the tissue forms produced using the device and methods described and contemplated herein are more consistent and precise than many other reshaping techniques and devices used for reshaping tissue samples. This is due to the various features of the device and available adjustments thereto, which enable increased control and consistency when reshaping tissue samples, as described in detail below.

The device and methods of its use which are described and contemplated herein may be used with, or applied to, tissue samples of any of various tissue types which may be recovered or produced from any of several sources. Although the sources of such tissue samples are typically mammals, other sources include, without limitation, reptiles, amphibians, and birds. Examples of mammalian sources for tissue samples include, but are not limited to: humans, primates, suines, bovines, ovines, equines, and canines.

Additionally, although suitable tissue samples will typically be autogenic or allogeneic with respect to their source, the tissue samples may also be xenogeneic. For clarity, autogenic tissue samples are recovered or produced from a source which is the same individual into which the resulting tissue form will be implanted. Allogeneic tissue samples are recovered or produced from a source which is a different individual, but of the same species, as the individual into which the resulting tissue form will be implanted. Xenogeneic tissue samples are recovered or produced from a source which is of a different species than the individual into which the resulting tissue form will be implanted.

The described and contemplated device and methods of its use may be effectively used with, or applied to, any of several types of tissues including, but not limited to, adipose, amnion, bone (e.g., cortical bone, cancellous bone, etc.), cartilage (e.g., hyaline cartilage, auricular cartilage, fibrocartilage, costal cartilage, etc.), chorion, dermis, fascia, intervertebral disc (e.g., annulus fibrosis, nucleus pulposus, etc.), intestine, ligament, liver, lung, muscle, pericardium, stomach, tendon, umbilical cord. Of course, tissue samples comprising a combination of one or more tissue types are also suitable for use with the device and methods of its use.

Generally, the device and methods of its use enable reshaping of tissue samples with increased control, precision and consistency. Reshaping tissue samples to produce tissue forms having desired sizes and shapes may be performed by any one or more of several methods and techniques such as, without limitation, cutting, shaving, grating, trimming, grinding, molding, curing, dehydration (e.g., air or heat drying, lyophilizing (freeze drying), etc.), contacting with one or more chemical substances, and crosslinking. The particular methods and techniques selected and applied often depend on the type of tissue being reshaped, as well as the desired size and shape of the tissue form to be produced. The desired size and shape of the tissue form may, in turn, sometimes be determined based on the intended use such as type, size, shape, and condition of a body feature, defect, or injury, to be treated, and the intended outcome the treatment.

As used herein, “size and shape” mean one or more of: overall size, one or more maximum dimensions (e.g., length, width, height, thickness, diameter, radius, etc.), one or more minimum dimensions (e.g., length, width, height, thickness, diameter, radius, etc.), linear contours, general overall shape, curved or arcuate contours, curvilinear contours, two dimensional and three dimensional geometric shapes, planar or sheets, surface texture or features (e.g., smooth, rough, wavy, raised bumps or projections, striations, channels, grooves, recesses, depressions, pores, cavities, etc.), and composites and combinations thereof.

The tissue type of a tissue sample being reshaped may have properties and characteristics that guide, determine, or dictate, which processing and reshaping techniques are effective, necessary, or both, to apply to a tissue sample to produce a tissue form having desired size and shape. Tissue properties and characteristics relevant to determining which processing and reshaping techniques would be useful and effective for reshaping a tissue sample include, for example without limitation, natural or initial shape after recovery but before reshaping, surface roughness, symmetry or asymmetry, rigidity, brittleness, plasticity, deformability, density, void space, elasticity, flexibility, mechanical strength (e.g., tensile strength, compressive strength, etc.), ductility, and rheology.

Some tissue types have one or more properties sufficient to withstand reshaping by cutting, shaving, grating, etc., to effectively produce tissue forms having desired sizes and shapes even with minimal or no external support or fixation. Bone tissue samples, for example, may generally be held in a vise or clamp during any one or more of cutting, slicing, shaving, and grating (or other reshaping techniques), to produce desired tissue forms such as, for example without limitation, monoliths, three dimensional geometric shapes, irregular three dimensional shapes, composite three dimensional geometric and irregular shapes, fibers, slices, sheets, particles, and combinations thereof.

On the other hand, some tissue types have one or more properties only partially sufficient to withstand reshaping by cutting, shaving, grating, etc., to produce tissue forms having desired size and shape. Dermis tissue samples, for example, generally have less rigidity and compressive strength, and more flexibility than, for example, bone tissue samples. Consequently, more external support and fixation of dermis tissue samples is sometimes necessary to perform one or more reshaping techniques (e.g., cutting, slicing, shaving, grating, etc.) and effectively produce tissue forms having desired size and shape. For example, a dermis tissue sample to be reshaped by cutting or slicing might rest upon a planar supporting surface and, may or may not additionally be held against the planar supporting surface by a pressure applying element (e.g., a plate, a bar, a template, etc.) which presses the surface of the dermis tissue sample opposite the planar supporting surface.

Many soft tissue types, including but not limited to dermis, cartilage, amnion, etc., are compressible to at least some degree which renders them difficult to reshape by techniques such as by cutting, slicing, grating, etc. This is because, when such soft tissue types are subjected to such reshaping techniques, compressive force is applied and such soft tissue types tend to deform under that compression. Furthermore, continued application of compression and other forces during efforts to cut, slice, grate, etc., the tissue sometimes tears. Accordingly, even when such reshaping techniques are partially effective, these tendencies to deform and tear when being cut, sliced, grated, etc., produce tissue forms having cut surface, edges, or both, which are not smooth or uniform, as is often desired and sometimes necessary.

The device and methods of its use described and contemplated herein are adapted to enable cutting or slicing a tissue sample along a single plane with increased control and precision to produce tissue forms having desired size and shape, as compared to many known cutting devices and guides and currently practiced cutting techniques. This, in turn, enables the production of tissue forms, whether intermediate or final, having consistent and precise sizes and shapes, especially with respect, but not limited, to thickness dimensions of the tissue form.

With reference to the drawings, an exemplary cutting device 10 for reshaping tissue samples by cutting or slicing will now be described. It is noted that some features of the exemplary cutting device 10 shown in the drawings are adapted to enable controlled, precise, and consistent reshaping of a surface and thickness dimension of a cartilage tissue sample to produce a cartilage tissue form having a naturally occurring first planar surface, a second planar surface formed by cutting or slicing, and a uniform thickness between the first and second planar surfaces. Nonetheless, it should be understood that the exemplary cutting device 10 shown in the drawings should not be interpreted to limit the features and operation of the cutting device 10, but rather, the design and principles of operation of the cutting device 10 described and contemplated herein may be used to reshape other types of tissue samples. Features of the cutting device 10 that are adapted for reshaping cartilage tissue samples to produce cartilage tissue forms as described above will be identified as such, and modifications thereto for accommodating other tissue types will also be explained below.

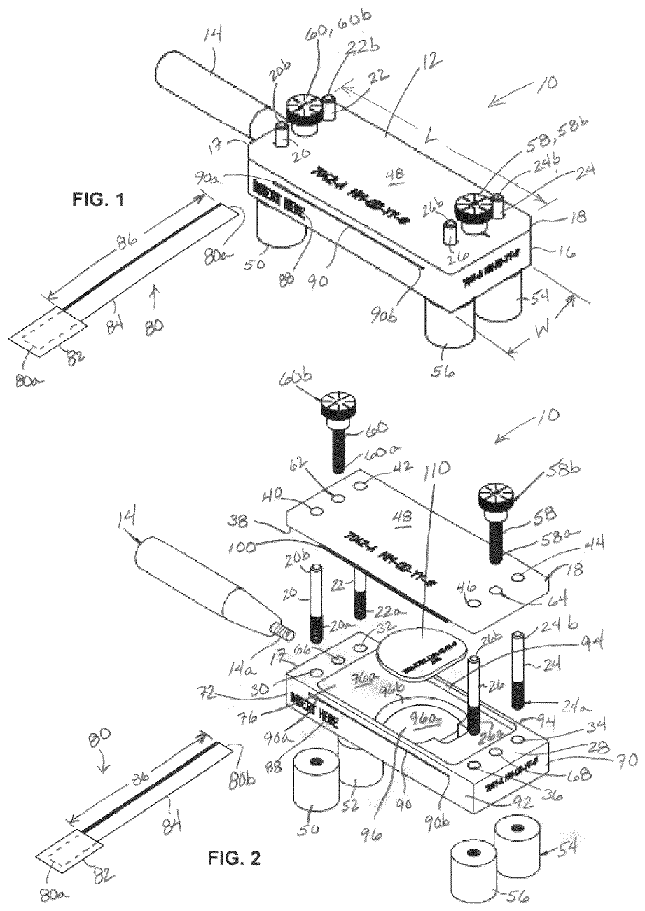

FIG. 1 provides a perspective view of the cutting device 10 in its assembled state, ready for operation by a user. FIG. 2 provides an exploded perspective view of the cutting device 10 which shows the several components and features of the cutting device 10, as will now be described in detail.

With reference initially to FIG. 1, the cutting device 10 generally includes a body 12 having a length L and a width W. A handle 14 is securely affixed to the body 12 for providing a safe and ergonomic place for a user to grip and hold the body 12 steady during operation of the cutting device 10. The body 12 includes a base plate 16 and a top plate 18 which is sized and shaped to fit on and be coextensive with the base plate 16. The handle 14 may have a threaded stud 14a at one end which is tightly screwed into a threaded bore (not shown per se) provided in a side wall 17 of the base plate 16.

Two or more stud guides, such as the four stud guides 20, 22, 24, 26 shown in FIGS. 1 and 2, are affixed to and extend upward from the base plate 16 for aligning and holding the top plate 18 in alignment with the base plate 16. In the exemplary embodiment shown in FIGS. 1 and 2, each stud guide 20, 22, 24, 26 has a threaded end 20a, 22a, 24a, 26a which is received and screwed into a respective one of two or more threaded bores 30, 32, 34, 36 in the top surface 28 of the base plate 16. The opposite end 20b, 22b, 24b, 26b of each stud guide 20, 22, 24, 26 is received through a respective one of two or more bores 40, 42, 44, 46, each of which extends though the top plate 18, from the bottom surface 38 to the top surface 48 of the top plate 18 (see FIGS. 2, 9, and 10).

With reference still to FIGS. 1 and 2, the cutting device 10 includes three or more stabilizing feet, such as the four rubber bumpers 50, 52, 54, 56 shown in the figures, each of which extends down from the base plate 16 proximate a respective corner thereof. The rubber bumpers 50, 52, 54, 56 provide contact points with a supporting surface, such as a table or desk (not shown per se), as well as resistance to minimize sliding by the body 12 of the cutting device 10 during assembly and operation.

The cutting device 10 also includes two or more knob screws 58, 60, such as a first (e.g., right) knob screw 58 and a second (e.g., left) knob screw 60, for moving the top plate 18 toward the base plate to apply downward force on the top surface 28 of the base plate 16. Each knob screw 58, 60 has a threaded end 58a, 60a which is received through a respective one of two or more through bores 62, 64, each of which extends though the top plate 18, from the bottom surface 38 to the top surface 48 thereof. After passing through the respective through bore 62, 64, each threaded end 58a, 60a of each knob screw 58, 60 is further received and screwed into a respective one of two or more threaded bores 66, 68 in the top surface 28 of the base plate 16. Each knob screw 58, 60 also has a knob head 58b, 60b positioned opposite the threaded end 58a, 60a and which may include texturing for increasing grippability.

In an exemplary embodiment, such as that shown in the figures, at least one knob screw 58, and the respective through bore 62 and threaded bore 66 therefor, are located proximate one end 70 of the body 12 of the cutting device 18a, and at least another knob screw 60, and the respective through bore 64 and threaded bore 68, are located proximate the opposite end 72 of the body 12 of the cutting device 10. This arrangement of the knob screws 58, 60 enables application of uniform or substantially uniform downward force across the top plate 18 when the knob screws 58, 60 are turned to the same degree as one another.

The cutting device 10 further includes a blade 80 having a blade holder 82 affixed at a first end 80a thereof for a user to safely hold and move the blade 80. The blade 80 has a linear cutting edge 84 and an exposed length 86 extending from (but not including) the blade holder 84 to the opposite leading end 80b of the blade 80. For reasons which will become apparent later, the exposed length 86 of the blade 80 is greater than the width W of the body 12 of the cutting device 10. Suitable blades 80 are flexible enough to resist breakage or snapping during use with the cutting device 10 and also have a thickness which permits smooth insertion and movement within the front and back slots 90 of the base plate 12 (only one of which is visible in the figures). Such blades 80 are, for example without limitation, commercially available, under Catalog #60-0153-0000, from AccueTec, which is headquartered in Verona, Virginia, U.S.A.

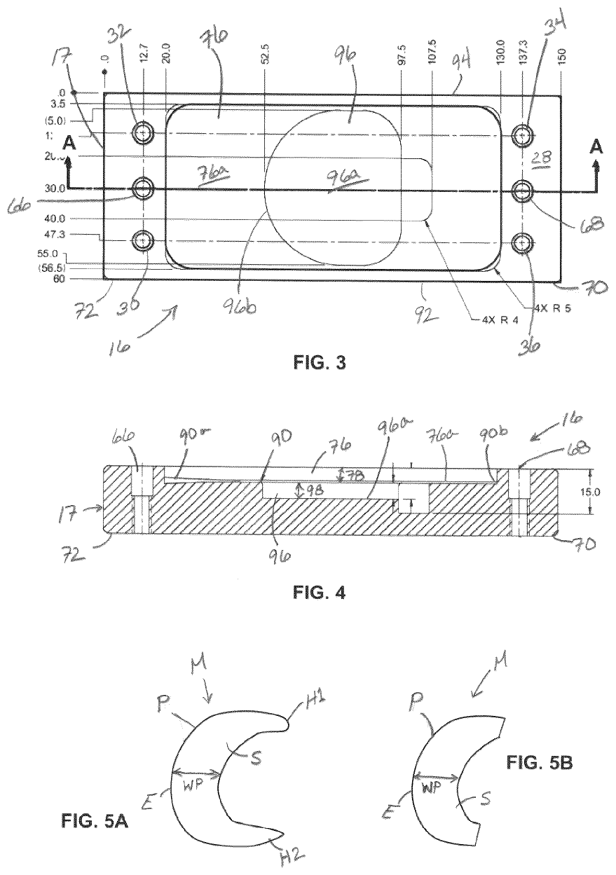

With reference still to FIGS. 1 and 2, the base plate 16 includes a horizontally oriented front slot 90 through the front wall 92 thereof, and a corresponding horizontally oriented back slot (not shown per se) through the opposite back wall 94 (see FIG. 3) of the base plate 16. The horizontally oriented front and back slots 90 (only one of which is visible) are sized and shaped as mirror images of one another and positioned on the front and back walls 92, 94 of the base plate 16. Furthermore, the horizontally oriented front and back slots 90 are positioned on the front and back walls 92, 94 in alignment with one another, for receiving the exposed length 86 of the blade 80 entirely through both of the aligned horizontally oriented front and back slots 90. The aligned front and back slots 90 also guide the blade 80 as it is moved in a horizontal direction by a user to perform a precise and consistent planar cut through a tissue sample, as will be explained below.

With reference now to FIGS. 2, 3, and 4, the base plate 16 includes a recess 76 in the top surface 28 of the base plate 16. The recess 76 has a uniform depth 78 which is measured from the top surface 28 of the base plate 16 to the bottom 76a of the recess 76. The recess 76 is shown in the exemplary embodiment of FIGS. 2 and 3 having a generally rectilinear shape as viewed from above, but may have any shape with a large open cross section such as, without limitation, an oval shape. Each of the horizontally oriented front and back slots 90 through the front and back walls 92, 94, respectively, of the base plate 16 is positioned to align with the bottom 76a of the recess 76 of the base plate 16.

The base plate 16 also includes a tissue cavity 96 which is sized and shaped for receiving and retaining a tissue sample therein (not shown in FIG. 2 or 3). The tissue cavity 96 is positioned entirely within the cross section of the recess 76 and has a uniform depth 98 which is measured from the bottom 76a of the recess 76 to the bottom 96a of the tissue cavity 96 (see FIG. 3). It is noted that the uniform depth 98 of the tissue cavity 96 determines the location of the planar cut which will, in turn, determine the thickness of a cut tissue sample product produced by operation of the cutting device 10. The movement and shifting of a tissue sample placed and held in the tissue cavity 96 is restricted and minimized, which facilitates cutting the tissue sample in a controlled, precise and consistent manner.

Since the exemplary embodiment of the cutting device 10 shown and described herein is designed for reshaping a meniscus (a cartilage tissue), whether a medial meniscus or a lateral meniscus, the tissue cavity 96 is sized and shaped to receive and retain such a meniscus therein. A meniscus is a cartilage tissue that may, for example without limitation, be recovered and separated from the knee of a mammalian donor. Cartilage tissue forms produced by reshaping a meniscus to have a substantially uniform thickness and substantially smooth and planar top and bottom surfaces are useful, for example, for performing surgical replacement and reconstruction of proximal interphalangeal (PIP) and metacarpophalangeal (MCP) joints. The cutting device 10 described and contemplated herein enables reshaping a meniscus (M) by making a precise and consistent planar cut into the thickness of the meniscus tissue sample (M) to produce the aforesaid desired cartilage tissue forms.

As shown in FIGS. 5A and 5B, a meniscus (M) typically has a natural crescent shape as viewed from above, with a posterior region (P) in between two ends, which are sometimes referred to as “horns” (H1, H2) (see FIG. 5A). As shown in FIG. 5B, the horns (H1, H2) may be cut off or otherwise removed prior to further reshaping using the cutting device 10. A meniscus (M) also typically has one crescent shaped surface which is substantially smooth and uniform, while the opposite crescent shaped surface(S) tends to be nonuniform and irregular, which means that the thickness across the meniscus (M) is also likely nonuniform. It is noted that only one crescent surface(S) of the meniscus (M) is visible in FIGS. 5A and 5B, and the above described differences in smoothness and texture are not per se visible in FIGS. 5A and 5B. Nonetheless, the aforesaid crescent shaped surfaces(S) of menisci and their properties are known and understood to persons of ordinary skill in the relevant art.

The widest span (WP) of the posterior region (P) is sometimes an important characteristic or parameter for the final tissue form and, therefore, may be measured and reported as the width of the meniscus cartilage tissue sample (M). It is noted that, as can be seen in FIGS. 5A and 5B, the posterior region (P) of a typical meniscus cartilage tissue sample (M) has an external arcuate edge (E). Accordingly, the tissue cavity 96 has a cross sectional shape which includes an arcuate edge 96b which at least partially conforms to the external arcuate edge (E) of the meniscus tissue sample (M). The tissue cavity 96 also has a uniform depth 98 which is sufficient to receive and hold a meniscus cartilage tissue sample (M) therein in a position which enables selection of the desired predetermined thickness of the reshaped meniscus (M) after controlled and precise cutting and removing of a portion thereof using the cutting device 10.

It should be noted that, although not shown in the present figures, the size and shape of the tissue cavity 96 may be modified to accommodate and/or at least partially conform to tissue samples of other tissue types and having different size and shape. For example, without limitation, a modified tissue cavity may have a shape with different contours, such as linear portions, or a concave arcuate edge, instead of the convex arcuate edge 96b of the tissue cavity 96 shown in FIGS. 2 and 3 and adapted for reshaping meniscus. Similarly, a modified tissue cavity may have a depth which is uniform or nonuniform, as desired, and which may be greater or less than the depth 98 of the presently described tissue cavity 96, depending on the expected thickness of the tissue samples to be reshaped.

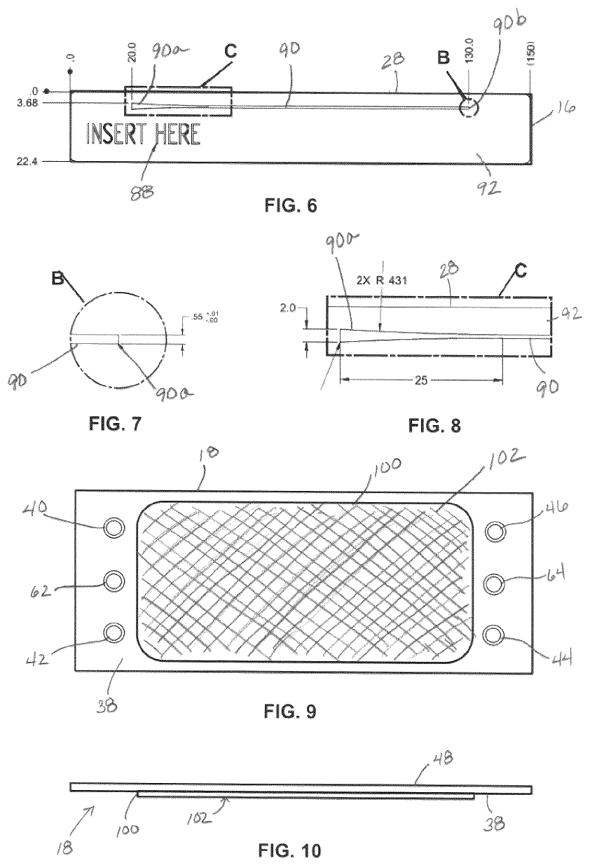

With reference now to FIGS. 6-8, the configuration and advantages of the horizontally oriented front and back slots 90 (only one of which is visible in the figures) through the front and back walls 92, 94 of the base plate 16 will now be described (see FIGS. 2 and 3 for back wall 94). FIG. 6 is a front elevational view of the base plate 16 of FIG. 3, showing the front slot 90 through the front wall 92 of the base plate 16 and having a beginning end portion 90a and a terminal end 90b. As shown in FIG. 6, text or other indicia 88 may be provided on the front wall 92 and proximate to the beginning end portion 90a to inform a user where the blade 80 should be inserted first during use of the cutting device 10.

FIG. 8 provides an enlarged view of region C of FIG. 6 which includes the beginning end portion 90a of the horizontally oriented front slot 90. As shown, the beginning end portion 90a has a tapered width for more easily inserting the blade 80 therethrough at the beginning of the cutting process. FIG. 7 is an enlarged view of region B of FIG. 6 which includes the terminal end 90b of the front slot 90. The horizontally oriented back slot (not visible in the drawings) through the back wall 94 of the base plate 16 is a mirror image of and is aligned with the front slot 96.

In an exemplary embodiment of the cutting device 10, the beginning end portion 90a is tapered, for a distance of about 25 mm, from a beginning width of about 2.0 millimeters (mm) to about 0.55 mm, and the remaining portion of the front slot 90 also has a uniform width of about 0.55 mm and extends to the terminal end 96b which is also about 0.55 mm wide.

With reference to FIGS. 9 and 10, the top plate 18 has the same dimensions as the base plate 16 and includes a raised layer 100 which protrudes from the underside or bottom surface 38 of the top plate 18. FIG. 9 provides a bottom plan view of the top plate 18, and FIG. 10 provides an elevational front view of the top plate 18. The raised layer 100 is sized and shaped to be securely received in the recess 76 of the top surface 28 of the base plate 16. Moreover, the raised layer 100 has a surface 102 with the same cross sectional size and shape as the recess 76. At least a portion of the surface 102 of the raised layer 100 contacts and applies force to a tissue sample (not shown) disposed in the cavity 96 of the based plate 16. To minimize lateral movement or shifting of a tissue sample disposed in the cavity 96 during the cutting process, the surface 102 of the raised layer 100 may have a rough texture or plurality of projections which grip or otherwise engage the upper surface of the tissue sample. This reduces or eliminates shifting and movement of the tissue sample (M) in the tissue cavity 96.

Due to the natural variability of tissue samples, the thickness of meniscus cartilage tissue samples may vary significantly enough to require adjustment to the depth 98 of the tissue cavity 96 in order to position the meniscus within the cavity 96 such that a planar cut into the thickness of the meniscus (M) will produce a tissue form having a smooth surface and uniform thickness. To make this adjustment, the cutting device 10 includes one or more spacers 110 (only one of which is shown in the figures), each of which is sized and shaped to fit into the tissue cavity 96 and lie flat on the bottom 96a of the tissue cavity 96. While each spacer 110 may have the same thickness such as, for example without limitation, 1 mm, and one or more such spacers 110 could be placed in the tissue cavity 96 depending on the size of the adjustment that is desired (i.e., three 1-mm spacers 110 would provide a total adjustment of 3 mm), it is believed that more precise and effective adjustments can be made where a plurality of spacers 110 having various different thicknesses are provided and available (e.g., a first spacer having a 1 mm thickness, a second spacer having a 2 mm thickness, and a third spacer having a 3 mm thickness, so that only a single spacer 110 which has a thickness equal to the size of the desired adjustment can inserted into the tissue cavity 96).

An exemplary method will now be described for using and operating the cutting device 10 to reshape a meniscus cartilage tissue sample (M) by making a planar cut to produce a cartilage tissue form having smooth top and bottom surfaces and uniform thickness therebetween.

The base plate 16, handle 14, stud guides 20, 22, 24, 26, and rubber bumpers 50, 52, 54, 56 are assembled together in the arrangements described above. Then, a meniscus (M) which has been cleaned and isolated is obtained and, optionally measured to determine the width of the widest span (WP) of the posterior region (P).

The meniscus (M) may be placed into the tissue cavity 96, smooth planar side down and in contact with the bottom 96a of the tissue cavity 96 and external arcuate edge (E) in contact with the concave arcuate edge 96b or the tissue cavity 96. Then, the meniscus (M) is visually inspected to determine whether the nonuniform surface of the meniscus (M) extends above the bottom 76a of the recess 76. If not, the meniscus (M) may be removed and one or more spacers 110 may be positioned in the tissue cavity 96, followed by placement of the meniscus (M) back in the cavity 96 on top of the one or more spacers 110.

The top plate 18 is then positioned above the base plate 16 and oriented to align the through bores 42, 44, 46, 48 with the stud guides 20, 22, 24, 26, followed by lowering the top plate 18 onto the base plate 16 so that the of the surface 102 of the raised layer 100 of the top plate 18 contacts and rests securely on the meniscus (M). The right and left knob screws 58, 60 are then inserted through the through bores 62, 64 of the top plate 16 and screwed into the threaded bores 66, 68 of the base plate 16, as previously described, at the same rate to apply downward force uniformly across the base plate 16 and meniscus (M).

A user then holds the handle 14 with their non-dominant hand and holds the blade 80 in their dominant hand. In cases where the user's dominant hand is their right hand, the leading end 80b of the blade 80 is inserted into the tapered beginning end portion 90a of the front slot 90, with the linear cutting edge 84 facing the meniscus (M) and the terminal end 90b of the font slot 90. Insertion of the blade 80 continues through the assembled body 12 of the cutting device 10 until the exposed length 86 of the blade 80 extends through the tapered beginning end portion of the back slot (not shown per se) which is aligned with the beginning end portion 90a of the front slot 90. As already may be deduced, in cases where the user's dominant hand is their left hand, the leading end 80b of the blade 80 is inserted into the tapered beginning end portion of the back slot (not visible in the figures), with the linear cutting edge 84 facing the meniscus (M).

After the blade 80 is inserted entirely through the body 12 of the cutting device 10 as described above, the user grips the handle 14 such that their thumb rests on top of the blade holder 82 and moves the blade 80 along the front and back slots 90 and through the meniscus (M) using a sawing motion. After the blade 80 passes entirely through the meniscus (M), thereby making a planar cut through the thickness of the meniscus (M), the blade 80 should be withdrawn from the body 12 and front and back slots 90. Both the right and left knob screws 58 and 60 are loosened at the same rate and withdrawn from the base and top plates 16, 18. Next, the top plate 18 may be lifted away from the base plate 18 and removed from the body 12 of the cutting device 10, followed by removing and inspecting the resulting meniscus cartilage tissue form from the tissue cavity 96. The cartilage tissue form produced by the foregoing method has smooth top and bottom surfaces and uniform thickness therebetween.

We claim:

1. A cutting device for precisely cutting a tissue sample with increased control and precision to produce a tissue form having desired size and shape, the device comprising:
a body which comprises:
a base plate extending between a first end and an opposite second end of the body, and having:
a top surface,
a recess having a recess bottom and an open cross-section and which extends, into the base plate, a recess depth which is measured from the top surface of the base plate to the recess bottom,
a front wall and an opposite back wall which form opposite sides of the open cross-section of the recess, each of the front and back walls extending between the first end and the second end of the body and from the top surface of the base plate to the recess bottom, wherein the base plate has a width defined by a distance between outer edges of the front and back walls,
a tissue cavity sized and shaped for receiving a tissue sample therein and which restricts and minimizes movement of the tissue sample thereby facilitating cutting the tissue sample in a controlled, precise and consistent manner, the tissue cavity being positioned within the recess and extending further into the base plate and having a cavity bottom and a cavity depth which is measured from the recess bottom to the cavity bottom, and
a cutting guide for receiving and guiding a blade while cutting a tissue sample disposed in the tissue cavity, the cutting guide comprising a front slot through the front wall of the base plate, and a corresponding back slot through the back wall of the base plate, the front and back slots having equal lengths, being mirror images of one another, and being oriented in alignment with one another and with the recess bottom in the base plate, and
a top plate which is sized and shaped to cover the top surface and the entire recess of the base plate by extending beyond the open cross-section of the recess; and
an elongate blade having a first end, an opposite leading end, a linear cutting edge extending from the first end to the leading end, and an exposed length of the blade comprising at least a portion of the cutting edge, wherein the exposed length extends farther than the width of the base plate for extending through both the front slot and the back slot, and wherein the elongate length of the blade has a width sized to facilitate lateral movement of the blade, along the lengths of the aligned front and back slots, from aligned beginning end portions to aligned terminal ends of the aligned front and back slots, which enables precise cutting, along a single plane, of a tissue sample disposed in the tissue cavity.

2. The cutting device of claim 1, further comprising a force applicator for moving the top plate toward the base plate to apply downward force on the base plate and against a tissue sample disposed in the tissue cavity, which restricts movement of the tissue sample during cutting, the force applicator comprising:
two or more knob screws, each of which includes a threaded end and a knob head on an opposite end,
two or more threaded bores, each extending into the top surface of the base plate and being sized and shaped for receiving and screwing therein the threaded end of a respective one of the two or more knob screws, wherein at least a first one of the two or more threaded bores is proximate to the first end of the body and at least a second one of the one or more threaded bores is proximate to the second end of the body, and
two or more through bores, each extending though the top plate and being sized and shaped for receiving therethrough the threaded end of a respective one of the two or more knob screws, up to but not including the knob head, wherein each of the two or more through bores is positioned on the top plate in alignment with a corresponding one of the two or more threaded bores of the base plate,
wherein, when each of the two or more knob screws is inserted through a respective one of the through bores of the top plate, and the threaded end of each of the two or more knob screws is received and screwed into a corresponding aligned one of the threaded bores in the base plate, turning the knob heads of the two or more knob screws, to a similar degree as one another, tightens the top plate against the base plate and applies substantially uniform downward force across the top plate and onto the base plate.

3. The cutting device of claim 1, wherein the recess depth is uniform across the recess.

4. The cutting device of claim 1, wherein the top plate includes a raised layer protruding from a bottom surface of the top plate, the raised layer having a bottom surface with a cross sectional size and shape for being securely and slidably received in the recess of the base plate and for enabling the bottom surface to contact and apply pressure to a tissue sample disposed in the tissue cavity.

5. The cutting device of claim 1, wherein the bottom surface of the raised layer of the top plate includes a tissue contact feature selected from a rough texture, a plurality of projections, or a combination thereof, for reducing or minimizing lateral movement or shifting of a tissue sample disposed in the cavity during cutting.

6. The cutting device of claim 5, wherein the tissue cavity is sized and shaped to receive and retain a meniscus cartilage sample.

7. The cutting device of claim 1, wherein the tissue cavity is sized and shaped to receive and retain a particular type of tissue sample, based on the dimensions of the particular type of tissue sample.

8. The cutting device of claim 1, wherein the tissue cavity has a cavity depth selected based on an expected thickness of a tissue sample to be reshaped.

9. The cutting device of claim 1, wherein the cavity depth is uniform across the tissue cavity

10. The cutting device of claim 1, wherein the blade further includes a blade holder affixed to the first end of the blade and covering a portion of the cutting edge to provide a safe place of the blade for a user to safely hold and move the blade.

11. The cutting device of claim 1, wherein each of the front and back slots is horizontally oriented on the front and back walls, respectively, of the base plate.

12. The cutting device of claim 1, wherein each of the aligned terminal ends of the aligned front and back slots, respectively, has and end width, which is sized to receive the exposed length of the blade therethrough, and wherein each of the aligned beginning end portions of the aligned front and back slots, respectively, has a beginning width greater than the end width for facilitating initial insertion of the leading end of the blade through the front slot and then through the back slot, prior to commencing cutting of a tissue disposed in the tissue cavity.

13. The cutting device of claim 12, wherein the beginning end portion of each of the front and back slots, respectively, is tapered from the beginning width down to the uniform width.

14. The cutting device of claim 1, further comprising one or more spacers for adjusting the cavity depth and thereby allowing controlled cutting of the tissue sample to produce a cut tissue having a desired tissue thickness, each of the one or more spacers having a spacer thickness and being sized and shaped to fit into the tissue cavity, lying flat on the cavity bottom and underneath a tissue sample when the tissue sample is disposed in the tissue cavity.

15. The cutting device of claim 1, further comprising an assembly guide for facilitating assembly of the body with the top plate aligned over the base plate in a position in which the top plate entirely covers the recess, the assembly guide comprising:
two or more elongate stud guides, each of which is affixed by a first end thereof to the base plate and extends upward, from the top surface of the base plate to an opposite free second end thereof, wherein at least a first one of the two or more elongate stud guides is proximate to the first end of the body and at least a second one of the one or more elongate stud guides is proximate to the second end of the body, and
two or more assembly bores, each of which extends though the top plate and is sized and shaped for receiving therethrough the second end of a respective one of the two or more elongate stud guides, wherein each of the two or more assembly bores is positioned on the top plate in alignment with a corresponding one of the two or more elongate stud guides.

16. The cutting device of claim 1, further comprising a handle which is securely affixed to one of the first end or the second end of the body and provides a safe and ergonomic place for a user to grip and hold the body steady during handling and use of the cutting device.

17. The cutting device of claim 1, further comprising three or more stabilizing feet, each of which is affixed at a first end thereof to and extends from a bottom of the base plate.

18. The cutting device of claim 1, further comprising four stabilizing feet, each of which is affixed at a first end thereof to and extends from a bottom of the base plate, wherein the base plate has a rectilinear shape and each of the four stabilizing feet is affixed proximate a respective corner of the base plate and has a rubber bumper on an end thereof which is opposite the first end for providing a slip resistant contact points with a supporting surface.
